(19) United States
(12) Patent Application Publication
(10) Pub. No.: US 2008/0274188 A1
(43) Pub. Date: Nov. 6, 2008

(54) Oral Vehicle For Systemic Pharmaceuticals

(75) Inventor: Carl Ernest Alexander, Auckland (NZ)

Correspondence Address: YOUNG & THOMPSON, 209 Madison Street, Suite 500, ALEXANDRIA, VA, 22314, US

(73) Assignee: PBL MANUFACTURING LIMITED, AUCKLAND (NZ)

(21) Appl. No.: 12/067,817

(22) PCT Filed: Sep. 22, 2006
(86) PCT No.: PCT/NZ2006/000246

§ 371 Date: May 20, 2008

(30) Foreign Application Priority Data
Sep. 22, 2005 (GB) 0519290.1

(51) Int. Cl. A61K 9/52; A61P 43/00

(52) U.S. Cl. 424/484

(57) ABSTRACT

Systemic drug delivery includes boluses of a semi-solid agar gel, each containing active ingredients, packed singly or in co-operating sets in blister packs, or loose in a container. When placed in the mouth the bolus is disrupted, comes apart, and releases the active ingredients. Sialogogues, flavours, and other additives assist in swallowing. Grains of the active ingredients may be encapsulated inside harder gel capsules. Active ingredients include over-the-counter medications and prescription medications. Applications include self-medication particularly in water—free situations such as public transport, medication for children, stroke victims or the aged.

### FIELD

This invention relates to systemic drug delivery means; a drug carrier for use with drugs to be administered by the oral route.

### DEFINITIONS

The term “BSSG” is used herein to refer to a bolus of undefined shape comprised of a semi-solid gel; each bolus to include an effective amount of at least one pharmaceutical or in the alternative a “taste masking agent” to follow administration of a bad-tasting medicine which may itself have been via a BSSG bolus. (BSSG=bolus: semi-solid gel).

The term “taste masking agent” is used to refer to any substance that can assist the act of taking a pharmaceutical substance, largely by reducing the bad taste perceived when some particular substances come in contact with taste buds in the mouth. Also, chasers such as water, disguisers such as flavouring agents or sweet substances, and coating agents such as honey or finely divided inert substances (see U.S. Pat. No. 6,998,139 as below), are included.

### BACKGROUND

This invention relates to systemic drug delivery means by the oral route or to intra-oral drug delivery. Most drugs are distributed for sale and use as dry tablets, pills, boluses or the like, these being single dose units and being dry, are relatively stable. Some medications are distributed as syrups, suspensions or other liquid medicines. For oral medications, absorption in the stomach and intestine is principally relied on for entry into the blood.

A commonly felt need is the need to swallow a dry tablet under circumstances where the customary rinse with a glass of water that helps swallowing is not available. A headache for example may arise at any time, such as when outdoors, or in an absence of wash-room facilities; or when no bottled water is carried; for instance when on a bus or train, when driving in traffic, in a theatre, in an important meeting, or when walking, and perhaps a headache or some other disorder is suddenly perceived to be coming on. Certain emergency medications, such as those for mitigating an anaphylactic response or for use in obstetrics may involve taking a systemic treatment orally, if no parenteral administration is available or feasible. Another is that of “treatment failure” as for children. Yet another problem stems from the hazards linked to poor swallowing of sizeable hard objects such as pills or capsules in particular patients, such as the elderly or the neurologically impaired with an impaired swallowing reflex. A further need relates to dosing pet or domestic animals with a medicine; it is an art to give a tablet to a cat with a successful and scratch-free outcome. Another need is supplying medication in emergency when parenteral administration cannot be provided or is inadvisable.

The oral cavity has been an acceptable route for painless drug delivery for many years. The mucosa is relatively permeable, has a rich blood supply, is robust, and recovers quickly if damaged. It has almost no cells of Langerhans, and is tolerant to possible allergens. Since the venous drainage does not pass through the liver, elimination of absorbed drugs is slower. Three possible sites within the buccal cavity include: sublingual, buccal and local sites. There is a possibility of accelerating uptake by iontophoresis or local massage. Yet is use is by no means universal.

### PRIOR ART

A patent search revealed EP 0651997 to Yamanouchi, which does describe the preparation of boluses for administration of medication through the buccal cavity, including 0.1-1.2 percent by weight of agar, made into capsule-free boluses which are distributed in blisters within blister packs. In contrast to the present invention, these boluses are largely comprised of 50-99% specified sugars—lactose and/or mannitol—and an additional procedure is used to dry out each bolus starting immediately after the agar sets, for an extended period of typically some hours or days, while the boluses are rendered hard enough to withstand being pushed through the rear of a blister pack. This is described as “a sufficient strength for the handling”. Hardness measurements (interpreted as crushing strengths) averaging around 2 or 2.45 kg (19.6-24 N) are provided. Raising the temperature of the agar solution, when first made, to nearly 100 degrees C. is considered important by the present inventor for improved properties of the bolus. EP 0651997 does not mention that aspect.

WO2004/037231 is an example of a group using capsules for medicinal use—in this case largely comprised of gum arabic and a water-soluble polymer inside a capsule. The present invention does not use gum arabic, and any capsule is non-essential. EP 0389700 described capsules including a plurality of soft agar-walled microcapsules, whereas the present invention describes homogenous boluses, not walled capsules. EP 0950402 described a chewable pharmaceutical with specifically a gelatin matrix, capable of being swallowed in less than 20 seconds—whereas the present invention uses agar, a different material with differing properties, and usually expects the material to be retained in the mouth for a much longer period. EP 1444975 to the present applicant disclosed a number of formulations for delivering toothpaste into the mouth and breaking up rapidly under applied force, so that the toothpaste can serve a mechanical function, whereas the present application intends that the formulations release their active ingredients more slowly so that diffusion occurs across the oral mucosa. U.S. Pat. No. 6,998,139 described a method for reducing bitterness of a bitter tasting drug taken as oral tablets, comprising immediately pre-coating the tongue with a finely divided inert material (such as titanium dioxide) which has the apparent effect of blocking off the taste receptors on the tongue. Multi-part quickly disintegrating dry tablets are claimed. Nuts from the area tree (betel nuts) are commonly chewed by South East Asians along with betel leaf and slaked lime, and are described as having a very bitter and sharp taste. We suspect that the slaked lime may serve the same masking function as the titanium dioxide of U.S. Pat. No. 6,998,139 although it is known to be used to extract the alkaloids (including arecoline) of the nut.

### OBJECT

It is an object of this invention to provide an alternative vehicle for administration of pharmaceuticals, or at least to provide the public with a useful choice.

### STATEMENT OF INVENTION

In a first broad aspect the invention provides an oral vehicle for carrying an effective amount of one or more desired ingredients (herein called “actives”) into the systemic circulation of a human or an animal by the oral route; the oral vehicle comprising at least one unit bolus (herein termed a BSSG (=bolus: semi-solid gel)), wherein the or each BSSG includes a matrix of a semi-solid gel comprised of agar or a functional equivalent thereof and carries an effective amount of one or more actives; the semi-solid gel having a hardness of from about 1 to about 15 Newtons per mm under test conditions defined herein and including from about 0.1% to over 5% by weight of agar.

“Actives” include without limit pharmaceuticals, antibiotics, medicines, vaccines, mineral and dietary supplements, health food supplements, plant extracts, placebos, alternative medicines, and materials voluntarily taken by a person.

Preferred medications or pharmaceuticals include (without limitation) medicines, antibiotics, oral vaccines, plant extracts, pharmacologically active peptides, vitamins, mineral and food supplements, (such as trace elements, including iodine), and placebos.

Example pharmaceuticals include (without limitation) the compounds known as ampicillin, cloxacillin, tetracycline, codeine phosphate, dextromethorphan, morphine, paracetamol (acetaminophen), nicotine, diclofenac, pholcodine, piperazine, pseudoephedrine, quinine, contraceptives, tadalafil, sildenafil, and substances serving as placebos; also antihistamines and/or adrenaline analogues for anaphylactic shock such as from bee stings.

More particularly preferred pharmaceuticals include those over-the-counter (non-prescription) materials for the suppression of pain, to overcome headache or migraine, as remedies for colds and influenza, to suppress nausea, and inhibit or kill protozoan parasites such as Plasmodium spp. (malaria); also vitamins, vitamin mixtures, trace elements and other health supplements; also breath fresheners, decongestants and suppressants for allergic reactions such as remedies for hay fever.

Preferably the mass of a single BSSG is about 0.6-1.2 grams, while larger sizes particularly those for intra-oral absorption may include up to about 2 grams of medication and up to about 3 grams of excipients, gels, flavours and the like.

Preferably the semi-solid gel is homogenous and includes agar or a functional equivalent thereof in an amount of from about 0.6% to 0.9% by weight of agar.

Alternatively the semi-solid gel is homogenous but encapsulated and the homogenous portion includes agar or a functional equivalent thereof in an amount of about 0.1% to 0.9% by weight of agar.

Preferably the oral vehicle comprises a set of at least two BSSGs each having a different set of actives; wherein a first BSSG is formulated so as to complement the formulation held within a second BSSG and thereby promote systemic absorption of the one or more actives held within the second BSSG when both BSSGs are taken at or about the same time.

In a related aspect, the at least two BSSGs are used to separately hold actives over a period of time; said actives being unstable if stored in the same BSSG over the period of time.

In one major option, the intended route of delivery is primarily that of gastrointestinal absorption after swallowing, facilitated by providing the bolus with at least one means capable in use of rendering the or each BSSG easier to swallow in the absence of water.

Preferably the promotion of systemic absorption is facilitated by means of at least one means selected from a range including: promotion of salivation, at least partial masking of adverse taste, providing a smooth exterior, and permitting the or each BSSG to be dissociated in the mouth before swallowing, so that an effective course of treatment will tend to be maintained.

In a second major option, the intended oral route of delivery is primarily that of intra-oral absorption over a period of time independently of swallowing, and promotion of systemic absorption is facilitated by means of at least one ingredient capable of eliciting at least one process selected from a range including: promotion of salivation, at least partial masking of adverse taste, supplying a surface-active agent, promotion of circulation within the oral submucosa, facilitating, by means of the properties of the semi-solid gel, the or each BSSG to be physically dissociated within the mouth, allowing diffusion to occur within the or each BSSG and/or providing a pleasant mouth feel (including providing a smooth exterior) so that an effective course of treatment will tend to be maintained. c) applied disruptive forces within the mouth (between the tongue and the teeth for example).

An optional supplementary physical means capable of promoting circulation within the oral submucosa comprises a toothbrush or the like.

Preferably the at least partial masking of adverse taste is caused by a process including at least one of: blocking of taste buds by including at least one coating substance, swamping the taste buds with sweetness by including at least one sugar or sweetener, or providing flavouring by including an effective amount of least one flavouring agent, thereby dominating the olfactory receptors.

In a subsidiary aspect, the taste masking agent is provided in a separate BSSG so that the person can manipulate timing of ingestion of the respective BSSGs in order to minimise an adverse taste of the at least one active. Alternatively the taste masking agent is supplied within the BSSG containing the actives.

Furthermore, at least some BSSGs further includes at least one of: a distinctive colouring agent, a distinctive opacifying agent, and a distinctive shape so that each of a range of types of BSSG is rendered distinctive in order to permit identification of at least one active held within.

Optionally the BSSG further includes a distinctive colouring agent in an amount sufficient to stain the interior of the mouth during and after use, so that uptake of the at least one active is confirmed.

Alternatively, a container of water is substituted for one BSSG; having an effect, when the oral vehicle is in use, of helping swallowing and/or of diluting a remaining bad taste, so that a course of treatment will be maintained.

Optionally, each BSSG is dipped in a substance capable of forming a relatively impervious seal over any exposed surfaces after cooling; the substance providing a less permeable material capable of serving as a coating that is less likely to permit outwards passage of active ingredients from the centre during storage.

In a second broad aspect, the invention provides a blister pack for holding an oral vehicle comprising sets of one or more BSSGs as claimed in any previous claim, wherein the seal materials of the blister pack are provided with frangible lines and tabs so that a person can peel a selected blister open and retrieve the contents without applying force to the contents.

In a third broad aspect, the invention provides a method for making a BSSG comprising the steps of assembling the raw materials in the advised amounts;

completely dissolving the agar, the salt, the saccharine, and the glycerol in the hot water;
dissolving or suspending the at least one active in the solution prepared at (b); optionally when cooled;
optionally dissolving at least one additive intended to facilitate swallowing in the solution prepared at (c);
optionally dissolving or suspending at least one dye in the solution prepared at (c);
dispensing the solution prepared at (e) into moulds each one holding an advised amount;
allowing the solution to solidify into a semi-solid gel and packing the resulting BSSGs in a container.

A method as claimed in claim 18 further including substances within the gel that are selected from a range including polyethylene glycols having a selected range of molecular weights, and polypropylene glycols having a selected range of molecular weights

A method as claimed in claim 19 wherein the method is adapted to include mechanical dispensing of the melted material prepared at (e) into a plurality of blisters for assembly into blister packs.

A method as claimed in claim 20 wherein the method is adapted to include mechanical dispensing of different melted materials having different compositions into each of a plurality of sets of blisters for assembly into blister packs: each set containing more than one distinctive BSSG.

Alternatively the method terminates with the extrusion of a nearly set mass into a cool environment and then chopping the extruded material into lumps of a predetermined mass.

A method as claimed in claim 18 wherein the method includes the steps of preparation and inclusion of microencapsulated ingredients that hold at last one active and/or flavours and/or colours.

In a related aspect the invention provides a kit of raw materials for low-volume use, wherein the kit is provided with an empty blister pack and seal, granular raw materials, dyestuffs and flavours in vials, and instructions so that a pharmacist can, by adding a prescribed medication and completing the process of creating boluses of a semi-solid gel, make up a specific course of medication to be administered in the form of BSSGs for a specific prescription.

In a fourth broad aspect, the invention provides a method of dispensing a BSSG to an animal wherein the BSSG is smeared inside the animal's mouth, adjacent the animal's cheek teeth (molars) so that the active or actives within the BSSG are absorbed within the animal's mouth.

### PREFERRED EMBODIMENT

The description of the invention to be provided herein is given purely by way of example and is not to be taken in any way as limiting the scope or extent of the invention. The terms “including” and “comprising” are both to be taken as not restricting the items in the accompanying list to solely those items identified in the accompanying list.

### DRAWINGS

FIG. 1: is a diagram showing a section through some typical BSSGs

FIG. 2: is a diagram showing a BSSG and a taste masking agent in a blister pack.

FIG. 3: shows how a blister pack is provided with an openable back.

FIG. 4: shows the deformation and fracture of a semi-solid gel according to the invention by applied force.

It would be desirable to have a more easily administered form of a wide range of “over-the-counter” or non-prescription medications, and prescription medications. Applications include self-medication particularly in adverse situations such as public transport, medication for children, the physically or mentally impaired, stroke victims or the aged. As will be explained below, the invention demonstrates utility in early trials without, as yet, using more sophisticated techniques such as microencapsulation of ingredients having a bad taste or requiring controlled release, or otherwise requiring further packaging.

Drug delivery means comprises lumps or boluses of a semi-solid gel, each (one BSSG) containing one dose of an amount comparable to that of a single solid tablet, including one or more active ingredients (or sometimes a flavour), packed singly in such as foil, or in blister packs, or loose in a container. When placed in the mouth the BSSG falls apart or dissolves over a period of time and releases the active ingredient(s). Sialogogues, flavours, and other additives assist in swallowing. Grains of the active ingredients may be encapsulated inside harder gel envelopes using the well-known procedures of micro-encapsulation. Active ingredients include “over-the-counter” medications and prescription medications. Applications include self-medication (particularly in adverse situations such as outdoors or in public transport), and medication for children, the physically or mentally impaired, stroke victims or the aged.

### EXAMPLE 1

The bolus (BSSG). Each “increment” of medication includes a pharmaceutically effective amount of a specified medication within a mass. Preferably but of course not essentially the amount in each BSSG is comparable to that of a single tablet of a pre-existing formulation, which avoids people making mistakes; always a possibility when a headache or other pain is active. The mass is a matrix of a semi-solid gel that retains its integrity during storage for a suitable period, and which is capable of being disrupted in the mouth when the bolus is administered. The bolus may include at least one additive having the effect, when ingested, of enhancing swallowing, although additives of that group are not essential components of the bolus. A single bolus may be made up by a pharmacist to hold more than one active ingredient, as long as the ingredients are mutually compatible during storage.

Although most compositions will be largely aqueous, a waterless formula having storage and/or release advantages may have only about 2% water with the remainder being made up of glycerol (glycerine), polyethylene glycol (PEG) of a selected average molecular weight range, or polypropylene glycol, (PPG) also of a selected average molecular weight range,

The gel of the BSSG. The semi-solid gel depends on the properties of agar, the gel being at a strength of about 0.8% agar in a bolus. The amount can be varied in order to make the bolus harder (with increased concentration, or softer as required. Furthermore, other materials included will affect the hardness and some can prevent setting. The 0.8% agar gel holds its shape if free-standing and will fracture if over-stressed. The shape is retained because the material is a “semi-solid” or soft solid. (The following paragraph measures what is meant by “semi-solid”.) In contrast, many products sold to the public as “gels” are semi-liquid rather than semi-solid, are based on polymethoxycelluloses and the like, and will flow without fracturing, like viscous liquids. The BSSG material has a “soft-melt” property in the mouth, when exposed to buccal temperature, saliva, and disruption by chewing, direct pressure, or other applied forces. Using agar, true melting is unlikely at body temperatures on account of the well-known hysteresis property of agar which means that it melts at a higher temperature than that at which it solidified. Preferably the toughness and softening point are selected so that the seeming “soft-melt” property is enhanced. Agar is currently preferred over the other naturally occurring gels. Artificial gels, or combinations of gels may be used. See the detailed recipe below. The type of agar used in trials is Coast Biologicals (NZ) food grade agar from seaweed.

### Quantifying “Softness/Hardness”.

This experiment allows the term “semisolid” as used herein to be approximately quantified. Tablet hardness meters (e.g. by Dr K Schleuninger AG of Zurich) apply an increasing force until the point of solid tablet disintegration is reached. The present test method measures the compression:force relationship before the gel under test gives way. Five representative samples were tested at room temperature, by using a controlled force to bring together two parallel surfaces encompassing the test sample. Force was measured using an electronic balance in air at about 35 deg C. and the compression caused was measured with a vernier scale to 0.05 mm. The force/distance relationship, reduced to Newtons to produce flattening by 1 mm was measured after waiting for creep to almost cease after each of 7 or 8 staircase-like increments of force, within an upper instrumental limit of 2.94 N. Results are provided in the following table. Sample 4 had partially dried after being stored for several years. Sample 5 was a day old.

TABLE 1
Measurements of softness of some semi-solid BSSGs.

| | Sample | Softness | |
|---|---|---|---|
| Sample | Dimensions | (N/mm) | Description |
| A (0.787 g) | Disk 14 mm dia, | 2.94 | Semi-soft, malleable, easily |
| | 5 mm high | | broken apart in the mouth |
| | | | or pressed by the tongue. |
| B (0.756 g) | Disk 12 mm dia, | 2.55 | As above |
| | 6 mm high. | | |
| C (1.085 g) | Disk about 14 | 1.13 | Soft to handle; Faster |
| | mm dia; 4 mm | | absorbtion. Creep continued |
| | high. | | when pressure >2.35 N |
| D (1.080 g) | Disk 14 mm dia, | 7.7 | Relatively tough yet still |
| | 5 mm high | | frangible in use. Slower |
| | | | absorbtion would be |
| | | | expected. |
| E (0.701 g) | Dome 14 mm dia, | 2.65 | Composition is that of |
| | 14.4 mm h | (0-1.6 N) | Example 1C. Fractured at |
| | | | 1.6 Newtons. |

These are mid-range rather than extremes in terms of a range of suitable hardness or softness. All samples included about the same percentage (0.8%) of agar. The hardness or softness of a given BSSG may be varied over a wider range than the above by varying the proportion of agar in the mixture from about 0.1 to 10%.

FIG. 4 shows a typical measurement sequence for sample E, as a graph 400 (wherein the linear portion of the plotted trace has been overlaid by a line 401, used to extract an average hardness value). At about 1.6 N the agar mass under test developed a radial crack and at just over 2 N the mass disintegrated into small cubes.

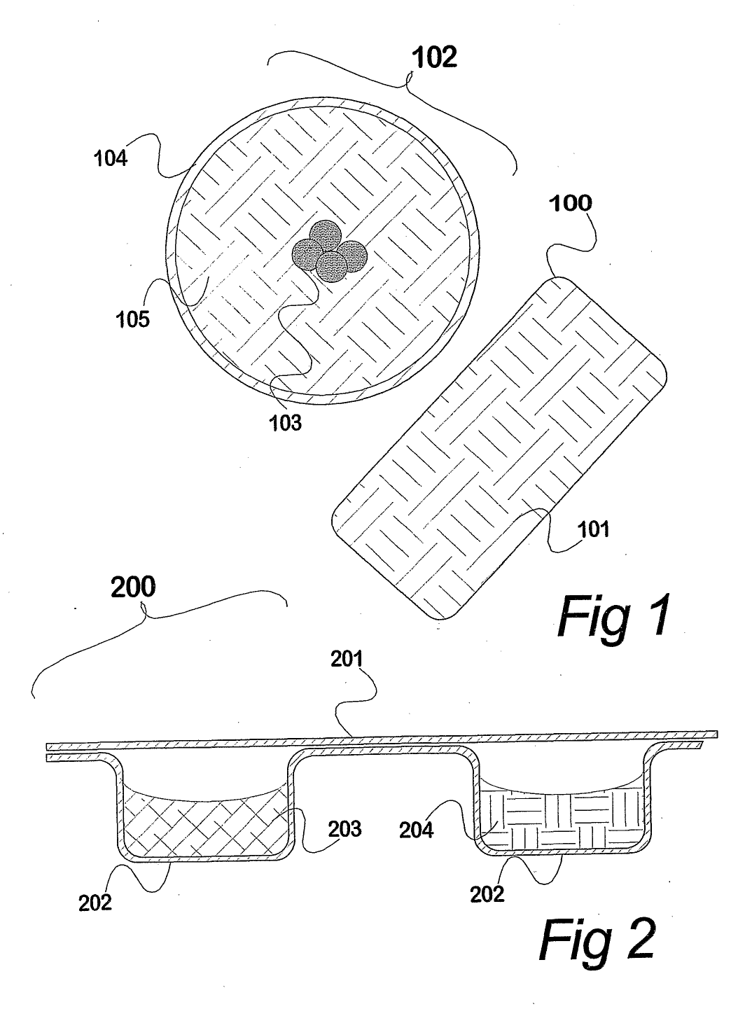

FIG. 1 shows at 100 a section through an approximately rectangular shaped BSSG. This example has no capsule or included material: the entire mass 101 is substantially homogenous and lacks a capsule although particles of a suspension, precipitate or suspended crystals may exist in some versions. FIG. 1 also shows at 102 a spherical version of a BSSG. This version includes an optional distinct capsule 104 as described below, a semi-solid gel mass 105, and an optional one or more inclusions of yet another material 103, which may be another pharmaceutically active material such as in capsules, or a mass of flavour-rich gel.

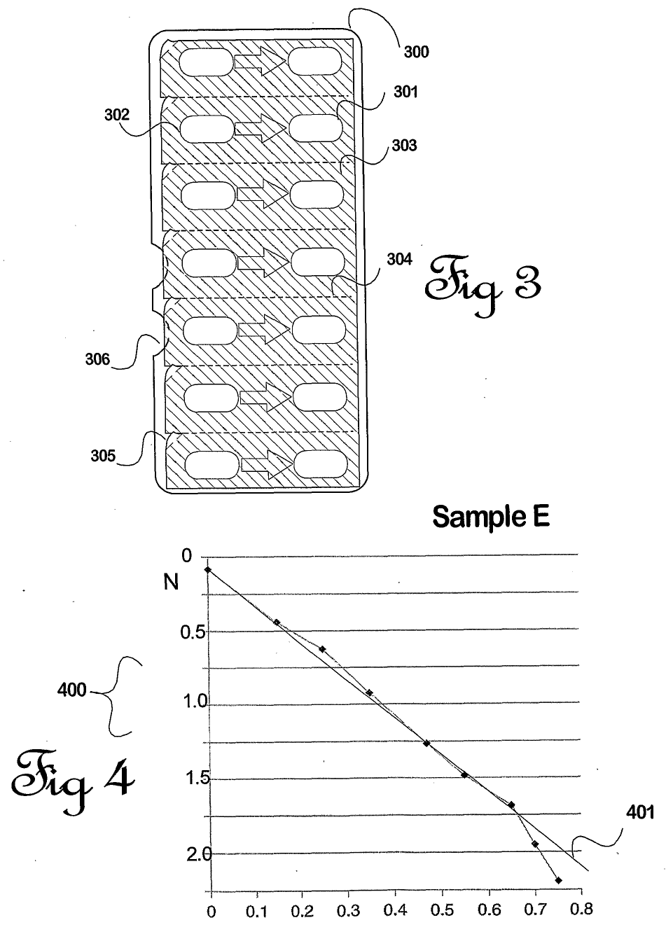

FIG. 2 is a section cut across a blister pack 200 having a cover 201 typically of a foil material detailed as in FIG. 3 and adherent in the usual way to a shaped plastics sheet including wells or blisters 202, each one containing a set-in-place material that was poured in while liquid; such as either a pharmaceutically active material 203 mixed into a solid gel or an inactive gel 204 including flavours—the “taste masking agent” mentioned below. The taste masking agent may be a more solid mass such as a piece of chewing gum, or the well may instead be filled with a second gel containing the same or another pharmaceutically active material.

Modifications. A tendency has been noted for some trial BSSGs made according to the general principle of a BSSG to “exude” an ingredient which then coats the exterior leads to a possibility that the first taste upon ingestion will be particularly bitter. This effect is a form of syneresis and is believed to be a result of the BSSG becoming supersaturated for the ingredient when cooled after formation. One solution is to add a step to the manufacturing process by dipping the BSSG in a less permeable coating; this may be another mixture of gels, a calcium salt of one or more alginate type gels, a gelatine coating, a wax coating, or a water-impermeable foil wrapping or impermeable pocket. A further solution is to use an ionic charge or the like to link the pharmaceutical to macromolecules of the gel or a modified gel or an added gel. Or, the concentration may simply be made lower.

Identification by appearance. Although there are a limited number of variants that can be applied to the bead itself, it would be desirable to separately identify at least each commonly used type of medication by appearance. It may be that only a small number of commonly consumed over-the-counter pharmaceuticals will be included in BSSGs according to the invention. Options available either singly or in combination (if not mutually exclusive on account of adverse drug reactions for example if unwisely mixed) include:

1. colour—with up to about 10 colour options:
a) the entire bead is the same colour; colour one or both ends with the same or different dyes or a pigment; internal granules are in one or more colours within a substantially water-clear bead.
   2. and/or opacity—
a) clear; translucent; opalescent (pearly); opaque; inadvertently, a glistening surface owing to syneresis and crystallisation for example with acetaminophen.
   3. and/or shape—
a) round; elliptical; rod-like (a shape which particularly lends itself to being divided if the person wants only a small dose); various blister-pack dictated shapes including cylindrical, oval, triangular, square, hexagon, pentagon star-shape or doughnut shape.

Any manufactured blister pack or portion thereof should of course be labelled by name, brand, batch and date in accordance with normal GMP practice.

Vehicle Destinations. At this point we shall differentiate between BSSGs as vehicles intended for facilitated swallowing and the site of absorption is gastro-intestinal, as against BSSGs as vehicles intended for trans-mucosal absorption within the mouth, although some BSSG formulations may be suited to both routes. Either form falls within the ambit of the invention. Both ways are likely to reveal the taste of the drug to the user.

In addition BSSGs including medicaments, drugs, pharmaceuticals, nutritional supplements, and the like herein referred to as “actives”) are supplied either (a) alone or (b) along with complementary BSSGs lacking actives but including substances aiding absorption, transport of the vehicle, or (c) along with different BSSGs including different and perhaps storage-incompatible actives.

### Compositions:

BSSGs suited to both routes include:
1. Water: up to over 90%. Long-term stability of specific actives in an effectively aqueous solution inside the BSSG should be considered, although water, being highly polar and physiologically compatible, has many advantages.
   a) Minimal water versions exist such as in Example 1D; II/122G which confers benefits in relation to storage and release.
2. Agar: 0.1-2%. At this point no other similar material appears to be as effective.
3. Actives, see Table 2 of exemplary deliverable pharmaceuticals, and note that peptides, hormones, Chinese medicines, food supplements, vitamins and even placebos are further classes of actives not included in Table 2.
4. Materials supporting particular actives, such as salts for osmotic and pH control, sugars, polymers such as polyethylene glycols and polypropylene glycols of suitable molecular weight ranges, excipient, including humectants (such as glycerol), salts for taste, sugars, saccharin's, carboxymethyl cellulose or other cellulose derivatives to enhance tackiness and adherence to gums, and the like.
5. Optional colorants and opacity-creating materials which along with shape (see above) serve to identify particular compositions.
6. Optional non-toxic dyestuff or a pigment, (additional to that required for identification) such as a food-grade dye to temporarily stain a person's mouth so as to indicate that a particular BSSG has been ingested properly. This is useful in some situations, such as in disaster medicine, when it is important to clearly show that a dose has been given to a poorly conscious patient or one unable to speak in a compatible language, or where patient management is not trivial, such as in a mental health ward.
7. Optional flavours for at least partial obstruction of a taste possessed by the or each active substance comprising the medication. They tend to be bitter in taste. Partial obscuration may be sufficient. It is widely known that strong medicines might taste bad and complete masking of that taste may have a psychologically mediated adverse effect on efficacy. Preferred flavours include menthol, peppermint oil, orange oil, and anise oil. For example, anise oil seems well suited to masking the bitterness of acetaminophen. PEG (polyethylene glycol) may mask adverse tastes.

Further, BSSGs as vehicles intended for facilitated swallowing may include:

Sialogogues, flavours, and other additives assist in swallowing—should the application be “swallowing away from water or other drinkable inert liquid”. Most sialogogues operate through taste and smell buds, and many suitable examples may be the flavours themselves, or salt (NaCl) or equivalent. The physical presence of the BSSG or fragments thereof in the mouth also act to promote salivation via reflexes including mechanoreceptors.

Combinations of “sub-vehicles” such as use of encapsulation as well as use of granules of harder agar within the overall soft agar bolus may assist carriage and slow the release of the active ingredient in the stomach.

Further, BSSGs as vehicles mainly intended for facilitated trans-buccal absorption may include:
1. The class of pharmaceuticals relatively capable of being absorbed by diffusion through the mucous membranes of the mouth and pharynx, and especially those which would be easily disrupted by stomach acidity had they been swallowed. Low-MW peptides such as oxytocin and perhaps insulin are included in this group of diffusible substances. A rising amount of products developed by the biotechnology industry is in the form of peptides. Oral vaccines may be included in this section.
2. Detergents and the like (as normally found in toothpastes) for which one application is for enhancing access through possibly thick saliva to the buccal epithelium.
3. Absorption-enhancing chemicals such as the class including dimethyl sulphoxide (DMSO). Ideally this would be provided within frangible microcapsules, but it is likely that they would have to be mixed into the BSSG soon before use.
4. Mild locally inflammatory substances (for increasing buccal blood flow); sialogogues such as the flavours themselves, or salt (NaCl) or equivalent. The physical presence of the BSSG or fragments thereof in the mouth also act to promote salivation via reflexes including mechanoreceptors and the presence of saliva will carry the actives around the mouth and reach a larger area of buccal epithelium, and initiate swallowing.
5. Substances for slowing the release of actives. Some active materials may diffuse out of the gel too quickly and for example fail to be absorbed, or overpower the taste buds or their masking means (flavours etc). The gel of each BSSG may be provided with an ionic charge by means (for example) of addition of a chemical material capable in one part of bonding to the gel and in another part of presenting appropriately charged portions, so that the pharmaceutical carried within the BSSG becomes indirectly bound to the interior gel.
6. Physical slowing (Encapsulation). The invention is suited to use of a “wrapping” stage wherein particles of the active medications are cloaked in a gel before mixing with the matrix, so that they are less likely to be tasted by the patient. “Particle” may include solids, or droplets, or oils. Two optional procedures are described below that will have the effect of reducing tasting by the patient. The cloaking gel may be an un-medicated material, a more concentrated agar, one treated to cause hardening, or may be an alginate hardened with calcium. Each cloaked gel particle may be about 0.1-1 mm in diameter.

The inventor believes that a way to overcome or at least render tolerable a bad taste is to provide another item such as a second BSSG holding some other substance or substances, herein called “taste masking agents” so that the person can ingest into the mouth a BSSG including the taste masking agent before, or with, or shortly after taking in the pharmaceutical-loaded BSSG (which would of course be differently identified such as by colour or shape, or by layout of the blister pack) and the taste masking agent would mask any remaining bad taste. The or all taste masking agent may be provided separately (not within the medicated BSSG itself) so that the person can delay ingestion until the active ingredient can be clearly tasted. Example taste masking agents include: water that serves as a diluent and as a “chaser”, a flavouring agent to dominate the olfactory receptors, a sweet material, or a taste bud coating material such as finely divided titanium dioxide (which does not appear to bind irreversibly to the pharmaceutical).

The accompanying bolus is not a necessary component of the invention but its presence aids the taking of medicines in those situations where some overcoming of a bad taste is involved, such as where no rinsing water is available. The alternative term for a taste masking agent—a “chaser” would imply sequential swallowing but it may be that the taste masking agent is held in the mouth after and/or while the primary BSSG is held in the mouth, so that a possible bad taste is neutralised after tolerating the taste for a limited period. Alternatively the taste masking agent may be taken first. The best order would be dependent on the particular compounds ingested.

Preferably the taste masking agent is a second BSSG made of different constituents; such as one including flavour or other taste-masking agents or supplies of water or a sialogogue, but (usually) lacking pharmaceuticals. (Sometimes a medication may endure storage if split into two parts). It is easy to manufacture mixed blister packs, by depositing a different BSSG material in different rows along the length of a blister pack, as a hot liquid that sets in place as a gel. This is technically simpler than placing a solid item in a blister. In some variations the taste masking agent may include a second pharmaceutical; one that would not survive storage if directly mixed with an incompatible first pharmaceutical. Indeed, there may be a further taste masking agent to help It may include a breath freshener that is not chemically antagonistic to the active pharmaceuticals. Preferably the active BSSG and the taste masking agent are clearly distinctive by appearance, so that a person who is confused or unwell would not confuse the two.

An alternative taste masking agent to a BSSG is for example a jelly bean; a boiled sweet, a lump of liquorice, breath-freshener, specially made product or chewing gum held in a blister alongside a blister holding the BSSG.

7. Active Medications and Ingredients in More Detail, with Examples.

We expect that most of those medications accepted for storage and delivery in syrups and other aqueous media such as suspensions will be sufficiently stable for inclusion in an aqueous gel as a BSSG. It is difficult to enumerate all the specific materials that are now or will be suitable for use in the future. In general it can be observed that it is easier to manufacture according to this recipe if the active ingredients are water soluble, although low-water versions of the BSSGs are described herein. Approaching conditions of a saturated solution may cause syneresis, and/or crystallisation during manufacture. Finely ground suspensions are acceptable if maintained in suspension during manufacture by recirculation (as is known in the art) until deposit in the BSSG.

Preferred medications or pharmaceuticals include (without limitation) medicines, antibiotics, oral vaccines, plant extracts, pharmacologically active peptides, vitamins, mineral and food supplements, (such as trace elements, including iodine), and placebos. Some specific examples are shown in the non-limiting Table.

TABLE 2
Some example deliverable pharmaceuticals, with CAS numbers and notes re applicability.

| Name | Purpose | Solubility water | Notes |
|---|---|---|---|
| Acetaminophen [103-90-2] | Analgesic, antipyretic, | Cold-v slight | Tends to form large crystals |
| OTC (panadeine, Panodol ™) | Maximum adult daily | Hot-considerably more | during manufacture. |
| | dose 4 grams a day | | |
| Quinine [30-95-0]] | Antimalarial | For the HCl: Cold-1 g | Substantially bitter. (Past |
| | | 16 ml | attempts to dispense it as dry |
| | | Hot-1 g 0.5 ml | tablets have resulted in the |
| | | | tablets passing through the |
| | | | body without absorbtion.) |
| Piperazine [110-85-0] | Anthelmintic | Freely soluble | |
| | (nematodes) | | |
| Pholcodine | Cough suppressant | 1 part in 50 | “very bitter taste” Dose up to |
| | | | 60 mg daily. |
| Morphine [57-27-2] | Analgesic | The HCl: Cold-1 g | |
| | | 17.5 ml | |
| | | Hot-1 g 0.5 ml | |
| Codeine phosphate [52-28-2] | Analgesic(narcotic) | 1 g in 2.5 ml water. | |
| Ampicillin [69-53-4] | Antibacterial | Sparingly soluble at | |
| | | room temp | |
| Tetracycline [60-54-8] | Antibacterial | The HCl: Freely soluble | Possible tendency for |
| | | | efflorescence from gel |
| | | | surface. Experimenting with |
| | | | much more mixing. |
| Cloxacillin (Na | Antibacterial | soluble | |
| monohydrate) [7081-44-9] | | | |
| “Multi-vitamin preparation” | Food supplement | | Gel presents natural brown |
| OTC | | | colour. |
| “MSM” thiol preparation for | Health food | Soluble- | made with black currant |
| joints OTC | | | flavour and blue colour |
| Dihydroergotamine | Anti-migraine | insoluble | Use a fine suspension. |
| Diclofenac [15307-86-5] | analgesic | | Include such as the synthetic |
| | | | prostaglandin “misoprostol”. |
| Sidenofil (Viagra ™) | Overcomes erectile | | Dose = 20-40 mg; easy to |
| | dysfunction | | include. |
| Antihistamines and decon- | Overcoming | | Consider maximising speed |
| gestants | anaphylaxis; | | of administration, if anaphy- |
| | overcoming allergies | | lactic shock. |

(OTC = over-the-counter; not a prescription drug.)

Precautions and Safety. Each pharmaceutical has its own problems in relation to storage and possible degradation. Drug interactions, and over-consumption should be guarded against, especially if the person taking the drug is a little dehydrated and not excreting much urine. Given that the invention is intended for a situation wherein ingestion otherwise requires the availability of water, packaging is in some cases likely to include the advice to take water or other liquid after ingestion of a BSSG bolus, where water or another drink is available.

Repeated doses of acetaminophen would warrant recommending an intake of water, just as for a person taking the drug in the form of gel caps or tablets. The moister nature of a BSSG as compared to a tablet would not overcome the need for water.

An example of a more difficult substance is diclofenac (Voltaren™) which is relatively toxic/irritative and has not been evaluated here. It is reported to “burn” the oral mucosa and, like aspirin, will cause stomach ulcers. This invention provides for use of such as synthetic prostaglandins in the gel of the BSSG for stomach protection, and micro-encapsulation of the diclofenac in 1-2 mm granules having a variety of wall thicknesses in order to delay its release to over a period after ingestion also after the prostaglandin has been released. Such techniques should overcome these problems if the market demand justifies development and testing. A suppository (a common route of administration for diclofenac) is not very easy to self-administer in a public place.

Children. One must guard against the possibility that a visually attractive container of flavoured semi-solid gels, left around, will be sampled by a child believing that they are sweets, and toxicity may arise. No risk reduction method is fully effective. Active parent control and use of a locked cupboard is the most secure protection. Some steps to minimise this form of risk include:
1. Dispensing BSSGs in sets, perhaps in a blister pack—typically with one tasting neutral or nice, intended for use as a “taste masking agent” (see later) having no active pharmaceuticals, and the other likely to taste nasty, including the active ingredients. Children would ingest only the nice tasting BSSGs, rejecting the nasty taste, and the pilfered sets alert the responsible adult to a problem of discovery.
2. Colours and appearances that are not linked to nice flavours—such as being unlike jellybeans.
3. Opaque packaging (such as by use of a titanium dioxide filler in the plastic material itself); also child-resistant packaging that is hard to open by toddlers and hard for them to remove the contents. Use of blister packs tends to make ingestion more difficult for a child.
4. Clearly one has to guard against a child or person of poor reasoning power consuming sweets or candies just for their nice taste and thereby accidentally ingesting a dangerous amount of a pharmaceutical. Supply of BSSGs within a blister pack is preferable over supply in bulk in a bottle for this reason. Few sweets are sold in blister packs. Young children find blister packs to be an obstacle. Any consumption is visible as emptied blisters. Protection of the contents from light (especially UV light) may also be required, and provided by dyes in the wall or foil wrapping.
5. Minimise the use of sugar or sweeteners in the BSSG in relation to the active ingredients.
6. Delay concealment of a nasty taste for a limited period in the mouth so that a child spits the BSSG out before any flavour becomes active. The flavour might be micro-encapsulated or otherwise bound so that its release is delayed.
7. Mouth coloration, if temporary oral dyes are included, will demonstrate what a potentially poisoned child has had.

The following Examples show methods for preparation of BSSGs according to the invention.

Storage. According to the invention the pharmaceutical is in contact with or dissolved in water; usually something considered to accelerate degradation over the dry state. Factors capable of extending storage life include (a) cooling or freezing, (b) the inherently immobile nature of the water in the gel, (c) use of excipients such as buffers and humectants, and (d) protection from light. The usual prudent storage of medications in blister packs kept in cool places should be recommended.

### EXAMPLE 1A

1. Method for preparation of BSSG including acetaminophen [103-90-2].

| | | |
|---|---|---|
| a) Agar | 0.8% | (all are % by weight) |
| b) Glycerol | 15% | |
| c) V41 (a type of sodium chloride) | 1% | |
| d) Sodium saccharine | 0.2% | |
| e) Boiling water | 42% | (Mix all) |
| f) Acetaminophen | 40% | |
| g) Anise Oil | 1% | (Add at a lower |
| | | temperature) |
| | = 100% | |

2. The mixture is cooled and mechanically divided or dispensed into preferably one gram BSSGs (any weight is of course possible: 100 mg to 2.5 g for instance) preferably in separate wells of a blister pack. Note that once the agar is dissolved at near 100 deg C., other components may be added to the still liquid mixture at considerably lower temperatures (such as 35-45 degrees) which is useful in the case of volatile flavours or easily thermally dissociated peptides.
3. Results: Colour: white. Anise oil provides a surprisingly effective mask for the bitter taste of the Acetaminophen. Each one gram BSSG holds 0.4 g acetaminophen. Max dose=about 2 g/day for an adult.
4. Notes: Some difficulty was experienced as a result of the acetaminophen tending to form large crystals while the solution was held and cooled while being dispensed into blisters. This may be overcome mechanically, as by using a centrifugal shearing type of recirculating pump and temperature maintenance, or by using an anti-crystallisation compound capable of inhibiting formation of crystals. Alternatively the mixture may be cast in temporary moulds and then transferred into blisters. Alternatively the acetaminophen may be added to the raw materials just before dispensing.

### EXAMPLE 1B

1. Preparation of BSSG including acetaminophen and ascorbic acid—for treating colds.

| | | |
|---|---|---|
| a) Agar | 0.8% | (all percentages are by |
| | | weight) |
| b) Glycerol | 15% | |
| c) V41 (a type of sodium chloride) | 1% | |
| d) Sodium saccharine | 0.2% | |
| e) Boiling water | 41% | (Mix and dissolve all |
| | | to this point) |
| f) Acetaminophen | 40% | |
| g) Tartrazine (or the lake of the dye) | 0.4% | |
| h) Orange oil | 1% | |
| i) Ascorbic acid | 0.2% | |
| j) Menthol | 0.4% | |
| | = 100% | |

2. The mixture is cooled and mechanically divided or dispensed into 0.5 to one gram weight BSSGs, preferably in separate wells of a blister pack.
3. Results: Colour: yellow. The orange oil and the ascorbic acid provide some perceived benefit for suffers from colds. Each 1 gram BSSG holds 0.4 g acetaminophen.
4. Taste as perceived: A half-BSSG of paracetamol made according to the above recipe including flavour was ingested without water. The material became fragmented into small parts within about 30 seconds and the combination of the foreign material and the flavour caused sufficient salivation for swallowing. A bitter after-taste was present for a few minutes but not at an objectionable level, and was of lesser perceived “intensity” than that of a typical headache.

Notes: Some initial difficulty was experienced as a result of the acetaminophen tending to crystallise when the solution cooled while being dispensed into blisters. This may be overcome mechanically, as by using a centrifugal shearing type of recirculating pump and temperature maintenance, or by using an anti-crystallisation compound capable of inhibiting formation of crystals. The acetaminophen may be added late. The mixture may be cast in temporary moulds and then transferred into blisters.

Since the active ingredients become available for absorption more quickly than is the case for encapsulated or dry compressed formulations, the person taking the BSSG may be advised to delay a second BSSG for 10 minutes rather than take two at once, so that the serum concentration, which will rise quickly, stays at an effective level for longer. Then the second BSSG may be found to not be necessary. Rather than be cast into blister or other moulds, the agar mixture may be allowed to flow into a cool oil so that globules of agar of about the desired volume are formed while suspended, and then sieved out. The BSSG may be extruded and formed as a long rod or ribbon to be cut up later.

### EXAMPLE 1C

Formulation made for stability trials of an earlier
developed formulation II/171D:1 kg White/translucent

| Agar | 0.8 (all % w/w) |
|---|---|
| Glycerine | 15.0 |
| Texapon OCN | 1.0 |
| Salt V41 | 1.5 |
| Sodium Saccharin | 0.1 |
| Boiling Water | 32.4 |
| Victor DF | 45.0 |
| Citric Acid monohydrate | 0.5 |
| Sodium monofluoro phosphate | 0.7 |
| Synthecol CAB | 2.0 |
| Peppermint B&J 684 | 1.0 |
| TOTAL | 100.0 |

### Specification:

Appearance—Crisp white gel with an oily surface.
pH of a 10% slurry in deionised water=5.5-6.0

### Manufacture:

1 Add agar, glycerine, Texapon OCN, salt, and saccharin to a jacketed pan capable of boiling water and mix.
2 Add boiling water to the pan and mix while raising the temperature to 95-100 deg C. Achieving this temperature is critical to the final product setup, and can be checked by ensuring that the mixture is absolutely clear and without opalescence. Turn off heat. (While such a high temperature is preferable the inventor realises that agar may be dissolved, albeit imperfectly, at lower temperatures).
3 Add Victor DF and citric acid and mix to a smooth white liquid.
4 In a separate unheated vessel mix the Synthecol CAB, and flavour and add to the heated mix when the temperature has fallen to less than 50 deg C. (Some components of a bolus may be unable to withstand temperatures over about 45-55 deg C., such as peptides or organisms inside vaccines).

### EXAMPLE 1D

### As 3 Versions

These variations comprise exploration of high-sucrose versions (II/122F), and low-water, high-glycerol versions (II/122G) both representing ways to affect the shelf life and the solubilisation rate of particular pharmaceuticals.

| Medicinal Bases referenced as: | II/122 D, | II/122 F | II/122 G |
|---|---|---|---|
| Agar | 4.0 | 2.0 | 5.0 |
| Glycerine | 30.0 | 15.0 | 90.0 |
| Salt V41 | 1.0 | 1.0 | — |
| Sodium Saccarine | 0.2 | | |
| Citric acid monohydrate | 0.2 | 0.2 | — |
| Sugar | — | 25.0 | — |
| Boiling water | 64.6 | 66.8 | 5.0 |
| TOTAL | 100.0 | 100.0 | 100.0 |

Manufacture yielded samples with the following properties:
II/122 D—Good set up, clear crisp gel. Also likely to work with polyethylene glycols, monopropylene glycol, and polypropylene glycols.
II/122 F—Inhibited set due to sugar, sets OK if sugar content lowered further.
II/122 G—Set improves as water content is raised above this level.

### EXAMPLE 1E

With granules separately prepared.

Example 1E may include a series of steps in order to prepare encapsulated ingredients, and the materials handling aspects should then ensure that the granules so formed remain evenly mixed and evenly dispensed.
a) Select a first gelling material. On account of syneresis which is a property of agar the first gelling material may not have the characteristic of a higher melting point than the gel used to form the bulk structure of the beads. In other words, the same composition of agar can be used at least twice. However it is preferable if the resulting pieces do not release unpleasant tastes in the mouth at least immediately, so further treatment is possible—see example 1B
b) Make a suspension of the medicament and mix it with the first gelling material after heating and liquefying.
c) Cool the resulting gel until it sets.
d) Break up the solidified gel mechanically into small pieces. (Here, techniques such as air beds for fluidizing are known). Freezing and sieving may be useful; controlling particle size will confer some control over the rate of release.
e) Dissolve and liquefy a second gel, which may include flavours and colours.
f) Mix the small pieces with the second gel and do not raise the mixture temperature above the softening level of the second gel, so that it stays intact during later processing.
g) The mixture is cooled and mechanically divided or dispensed into BSSGs each of a predetermined mass, such as 0.5 to one gram boluses. Larger boluses may be used since it is not difficult in most circumstances to swallow 2 g or more, although some persons who have difficult in swallowing would present problems.

### EXAMPLE 1F

### Like 1E, with Encapsulation of the Separate Granules

Example 1A may be modified by adding more steps between steps d and e as follows:

d1) As a third gelling material, select one that may be hardened; for example sodium alginate.

d2) Dissolve and liquefy a sodium alginate gel.

d3) Immerse the pieces of either (a) ingredients, or (b) ingredient-containing gel in the sodium alginate gel, coat them, and bring them out again.

d4) Harden the coating by conversion into calcium alginate gel, by dipping the gel into a calcium chloride brine, so that the coating will stay intact during a later period when held in the mouth.

### EXAMPLE 2

### Packaging

The inventor considers that packing the BSSGs into an ordinary blister pack is a clean, convenient and safe way to prepare the product for retail presentation, also suitable for the user to then carry around the medication in appropriate amounts that are always ready for use. It is easier to handle the materials in a factory as a liquid to be poured hot than by placing solid items (tablets) individually into blisters. A technology for carrying out a hot-pour process of molten agar into a blister pack includes the steps of:

(1) mixing the active ingredients in a vat and keeping them hot. The mixture shall be made with adequate accuracy and to the usual (GMP) standard required of a pharmaceutical factory.

(2) dispensing the mixture from a volumetrically accurate dispensing device into open blisters

(3) cooling and then sealing the blisters. (Foil wrapping may also be included).

(4) packing the blisters such as in cardboard boxes suitable for retail sale.

A blister pack protects the BSSG until it is taken for ingestion, carries appropriate labelling, and an empty blister indicates that a BSSG has been used. A throughput of well over a hundred thousand blisters an hour can be achieved on a production line, without direct human involvement.

Loose BSSGs of approximately globular form may be made by dispensing as large globules into an oil and sieving the boluses out when cooled and solid.

BSSGs may be made in a variety of shapes including round, square, triangular, star, disk, rod, plate, and these shapes may be determined by the blisters forms in the blister pack.

Note that the BSSG according to presently preferred formulations is too soft to act as its own break-out device when being pushed out of a sealed blister pack. For that reason the inventor prefer to provide blister packs having turned-up, adhesive-free edges on the rear (flat, non-blister) surface which may be pulled open by the user at the time of use, and/or perforations to assist in the removal of one or more BSSGs as required without destroying their structure.

FIG. 3 shows an example 7×2 pocket blister pack 300 from the rear (flat) side, and the hatched portion 303 of the diagram indicates that part of the backing sheet, which is usually a metal foil, that is selectively coated with an adhesive. Pockets such as 301 and 302 may contain distinct types of BSSG. For the purpose of opening the blister pack without pressing through from the front side, this pack is provided with (a) relatively tacky, removable adhesive, (b) perforations or functional equivalents (such as 304) that allow the backing to be removed in strips, and (c) a free corner 305 that the person would pull on first, to start peeling the back off along one row. The arrow 306 is printed on the backing foil merely to indicate the direction in which to remove the contents. The deformed plastic sheet including blisters 300 may also be provided with a pressed-down indentation beneath each tab 305 so 590 that the user can more easily grasp the free edge. Notch 306 cut into the side of the deformed plastic sheet illustrates a further way of preparing an easy-to-open portion of a sealed blister pack. Other foil modifications that facilitate opening and recovery of a soft gel interior may also be used. The inventor prefers that the deformed plastic sheet is made of a relatively strong material (such as 0.5 mm polyvinylchloride or PVC) in order to provide mechanical protection for the BSSG during long-term carriage in a pocket or the like. This material is supplied 595 coated with polyvinylidine (PVDC) at various thicknesses for an effective water barrier. For example, 0.25 mm PVC plus 40 grams per square metre (gsm) PVDC for storage inside a box, or 0.5 mm PCV plus 80 gsm PVDC for durable storage in a loose assemblage. to be selected according to effective shelf life requirements and the thickness of the base plastic. The deformed sheet may also be made of foil, such as for military use.

### EXAMPLE 2a

### Packaging without Blister Pack

Despite the benefits of a blister pack for many purposes, BSSGs may be made loose, without a blister pack. For example they could be extruded from an orifice of controlled shape into cold air or cold oil, and cut off from the orifice when of a suitable length. They could be patted dry of oil, or left to drip, and could then be packed in a box or sealable jar or other container together with a powder such as comflour to prevent sticking together.

### EXAMPLE 2b

### Packaging to Prescription by a Pharmacist

A kit for making batches of BSSGs is supplied as raw materials lacking only specified pharmaceutical(s) so that a pharmacist can make up a specific course of medication to be administered as BSSGs as set down by prescription for a specific case. The kit would include one or more sets of: an empty blister pack and seal, a plastic squeeze bottle, agar powders, dyestuffs and vials, and instructions. The required apparatus is a heated jacket device with hot water maintained at about 95-98 deg C. so that the agar can be heated sufficiently during melting.

### APPLICATION EXAMPLE 1

Administration of medication to overcome an immediate problem is a useful application. For example the treatment of the more severe allergic reactions, classical anaphylactic shock or an accident such as a sting from a wasp in the mouth, especially where the pharynx has swelled but nobody has or is capable of administering parenteral (e.g. intravenous) medication may be done using a BSSG containing a suitable antihistamine in an affective amount. The BSSG is pressed against the inside of the mouth and uptake may be encouraged by using a disposable toothbrush, provided in the kit, to rub the oral mucosa or the gums so that the surface is fresh and the circulation of blood is enhanced. After all, there is still a squamous epithelium to be passed through before the drug gains access to the blood supply. An antihistamine plus taste masking agent plus toothbrush kit would help many kinds of emergency worker (such as first-aid workers, in-shore coastguards, police, teachers, ambulance people and the like to respond to a medical emergency and lower the need for urgent proper medical treatment by a doctor who should have injectable adrenaline (epinephrine) with him/her. Persons prone to severe allergic reactions are also likely to carry injectable adrenaline.

### APPLICATION EXAMPLE 2

Nicotine replacement therapy for smokers may be provided by means of a BSSG holding an effective amount of from 2 to 8 mg of nicotine, so that the (ex) smoker can experience the effects of nicotine therapy but others are not subjected to adverse effects of smoke. In this example, a number of BSSGs each holding a suitable amount of nicotine are supplied, probably as relatively tough plates in a blister pack. The user takes one and holds it between the gum and the cheek for about 20 minutes, during which time the BSSG slowly melts away. That slowness helps limit repetition of self-dosing with nicotine.

### Variations

The invention may be used when treating domestic animals with oral medications. A commonly found problem is the difficulty of treating a cat (in particular) with a tablet and usually a treatment course fails to be given when a cat is sent home from a veterinary surgeon with a course of tablets. A soft bolus, that may be broken up against the animal's teeth along the inside of the mouth—on to the molars—(and optionally the bolus is provided with an appealing flavour such as fish) may be easier to administer to most cats than a hard dry tablet or capsule.

### INDUSTRIAL APPLICABILITY AND ADVANTAGES

1. A BSSG can be cut into parts with a pair of scissors for example, if a person does not require a full dose, because the body of the preferred type of BSSG is homogenous. (A capsule cannot be cut into parts, although a solid tablet can be snapped along the crease line).
2. The delivery means is advantageous for some pharmaceuticals and some diseases. For example, a quicker, higher peak in serum concentration of acetaminophen is expected, as compared to ingestion of a tablet or capsule. This faster rise is an advantage to a person wanting quick relief for example from a headache.
1. Smaller doses may be effective if taken by the BSSG route, reducing the risk of cumulative toxicity. One dose might work on its own.
2. A BSSG containing a finely divided solid material will allow the material to be absorbed and become available more quickly than one ingested within a capsule such as one of gelatine, which first has to be dissolved in the stomach. A rise in the blood levels of the ingested pharmaceutical should be steeper if administered within a BSSG.
3. In an emergency situation, where a patients needs a systemic drug and cannot be moved or held upright to swallow ordinary tablets, and in particular where helpers are not able or trained to administer injected materials, the BSSG type medication could be administered to be broken apart either by an assistant or within the patient's mouth, and be swallowed slowly and absorbed over time, with minimal risk of choking the victim or patient.
4. The invention is intended to help where there is a need to swallow a treatment held in solid form under circumstances where the customary accompanying glass of water is not available—such as when outdoors (walking or sailing), on a bus or train, when driving in traffic, in a theatre, in an important meeting, or when walking, and perhaps a headache of some other disorder is suddenly perceived to be coming on. Care to avoid adverse effects such as an increased risk of toxicity if the BSSG is taken without water, simply because of lowered kidney and/or liver function. The same effects would occur with a dry tablet, but the nature of the BSSG or the way that it is promoted may inadvertently suggest to a user that water is not necessary.
5. Another application is that of “treatment failure” as for children. Yet another is based on the hazards linked to poor swallowing in the elderly or neurologically impaired.
6. The invention is also suitable for giving medication to pet animals or birds, and to farmed animals.
7. The invention complements other forms of drug delivery such as oral, rectal, pulmonary, or parenteral methods.

Finally, it will be understood that the scope of this invention as described and/or illustrated herein is not limited to the specified embodiments. Those of skill will appreciate that various modifications, additions, known equivalents, and substitutions are possible without departing from the scope and spirit of the invention as set forth in the following claims.

1. An oral vehicle for carrying an effective amount of one or more active ingredients (herein called “actives”) into the systemic circulation of a human or an animal by the oral route; the oral vehicle comprising at least one unit bolus (herein termed a BSSG (=bolus: semi-solid gel)), characterised in that the or each BSSG includes a matrix of a semi-solid gel comprised of agar or a functional equivalent thereof and carries an effective amount of one or more actives; the semi-solid gel having a hardness of from about 1 to about 15 Newtons per mm under test conditions defined herein and including from about 0.1% to about 10% by weight of agar.

2. A BSSG as claimed in claim 1, characterised in that the semi-solid gel is homogenous and 680 includes agar or a functional equivalent thereof in an amount of from about 0.6% to 0.9% by weight of agar.

3. A BSSG as claimed in claim 1, characterised in that the semi-solid gel is homogenous but encapsulated and the homogenous portion includes agar or a functional equivalent thereof in an amount of about 0.1% to 0.9% by weight of agar.

4. An oral vehicle using BSSGs as claimed in claim 1, characterised in that the oral vehicle comprises a set of at least two BSSGs each having a different set of actives; wherein a first BSSG is formulated so as to complement the formulation held within a second BSSG and thereby promote systemic absorption of the one or more actives held within the second BSSG when both BSSGs are taken at or about the same time.

5. An oral vehicle using BSSGs as claimed in claim 4, characterised in that the at least two BSSGs are used to separately hold actives over a period of time; said actives being unstable if stored in the same BSSG over the period of time.

6. An oral vehicle using BSSGs as claimed in claim 2, characterised in that the intended route of delivery is primarily that of gastro-intestinal absorption after swallowing, facilitated by providing the bolus with at least one means capable in use of rendering the or each BSSG easier to swallow in the absence of water.

7. An oral vehicle using BSSGs as claimed in claim 6 characterised in that the promotion of systemic absorption is facilitated by means of at least one means selected from a range including: promotion of salivation, at least partial masking of adverse taste, providing a smooth exterior, and permitting the or each BSSG to be dissociated in the mouth before swallowing, so that an effective course of treatment will tend to be maintained.

8. An oral vehicle using at least one BSSG as claimed in claim 2, characterised in that the intended oral route of delivery is primarily that of intra-oral absorption over a period of time independently of swallowing, and promotion of systemic absorption is facilitated by means of at least one ingredient capable of eliciting at least one process selected from a range including: promotion of salivation, at least partial masking of adverse taste, supplying a surface-active agent, promotion of circulation within the oral submucosa, facilitating, by means of the properties of the semi-solid gel, the or each BSSG to be physically dissociated within the mouth, allowing diffusion to occur within the or each BSSG and/or providing a pleasant mouth feel (including providing a smooth exterior) so that an effective course of treatment will tend to be maintained, c) applied disruptive forces within the mouth (between the tongue and the teeth for example).

9. An oral vehicle using at least one BSSG as claimed in claim 8, characterised in that a supplementary physical means capable of promoting circulation within the oral submucosa comprises a toothbrush.

10. An oral vehicle using at least one BSSG as claimed in claim 7, characterised in that the at least partial masking of adverse taste is caused by a process including at least one of: blocking of taste buds by including at least one coating substance, swamping the taste buds with sweetness by including at least one sugar or sweetener, or providing flavouring by including an effective amount of least one flavouring agent, thereby dominating the olfactory receptors.

11. An oral vehicle including at least one BSSG as claimed in claim 10, characterised in that the taste masking agent is provided in a separate BSSG so that the person can manipulate timing of ingestion of the respective BSSGs in order to minimise an adverse taste of the at least one active.

12. An oral vehicle including at least one BSSG as claimed in claim 11, characterised in that the taste 725 masking agent is supplied within the BSSG containing the actives.

13. A BSSG as claimed in claim 1, characterised in that the BSSG further includes at least one of: a distinctive colouring agent, a distinctive opacifying agent, and a distinctive shape so that each of a range of types of BSSG is rendered distinctive in order to permit identification of at least one active held within.

14. A BSSG as claimed in claim 1, characterised in that the BSSG further includes a distinctive colouring agent in an amount sufficient to stain the interior of the mouth during and after use, so that uptake of the at least one active is confirmed.

15. An oral vehicle based on BSSGs as claimed in claim 9, characterised in that a container of water is substituted for one BSSG; having an effect, when the oral vehicle is in use, of helping swallowing and/or of diluting a remaining bad taste, so that a course of treatment will be maintained.

16. A blister pack for holding an oral vehicle comprising sets of one or more BSSGs as claimed in claim 1, characterised in that the seal materials of the blister pack are provided with frangible lines and tabs so that a person can peel a selected blister open and retrieve the contents without applying force to the contents.

17. A BSSG as claimed in claim 1, wherein each BSSG is dipped in a substance capable of forming a relatively impervious seal over any exposed surfaces after cooling; the substance providing a less permeable material capable of serving as a coating that is less likely to permit outwards passage of active ingredients from the centre during storage.

18. A method for making a BSSG comprising the steps of a) assembling the raw materials in the advised amounts; b) completely dissolving the agar, the salt, the saccharine, and the glycerol in the hot water; c) dissolving or suspending the at least one active in the solution prepared at (b); optionally at a cooler temperature:
d) optionally dissolving at least one additive intended to facilitate swallowing in the solution prepared at (c); e) optionally dissolving or suspending at least one dye in the solution prepared at (c); f) dispensing the solution prepared at (e) into moulds each one holding an advised amount; g) allowing the solution to solidify into a semi-solid gel and packing the resulting BSSGs in a container.

19. A method as claimed in claim 18 further including substances within the gel that are selected from a range including polyethylene glycols having a selected range of molecular weights, and polypropylene glycols having a selected range of molecular weights.

20. A method as claimed in claim 19 wherein the method is adapted to include mechanical dispensing of the melted material prepared at (e) into a plurality of blisters for assembly into blister packs.

21. A method as claimed in claim 20 wherein the method is adapted to include mechanical dispensing of different melted materials having different compositions into each of a plurality of sets of blisters for assembly into blister packs: each set containing more than one distinctive BSSG.

22. A method as claimed in claim 18 wherein the method includes the steps of preparation and inclusion of microencapsulated ingredients that hold at least one active and/or flavours and/or colours.

23. A method as claimed in claim 18 wherein the method terminates with the extrusion of a nearly set mass into a cool environment and then chopping the extruded material into lumps of a predetermined mass.

24. A kit of raw materials for low-volume use, wherein the kit is provided with an empty blister pack and seal, granular raw materials, dyestuffs and flavours in vials, and instructions so that a pharmacist can, by adding a prescribed medication and completing the process of creating boluses of a semi-solid gel, make up a specific course of medication to be administered in the form of BSSGs for a specific prescription.

25. A method of dispensing a BSSG to an animal wherein the BSSG is smeared inside the animal's mouth, adjacent the animal's cheek teeth (molars) so that the active or actives within the BSSG are absorbed within the animal's mouth.
